SEQUENCE LISTING (5 sequences)

SEQ ID NO: 1 - protein, 40 aa, Artificial sequence
  consensus sequence, in which the specific amino acids at positions 3, 12, 14, 15, 18, 21, 25, 34 and 38 are spaced by any amino acid
XXCXXXXXXXXDXLLXXCXXCXXXCXXXXXXXXCXXXCXX

SEQ ID NO: 2 - protein, 348 aa, Artificial sequence
  this is a fusion protein sequence containing a portion of a human TACI receptor and a portion of a human immunoglobulin heavy chain sequence.
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1044 bp, Artificial sequence
  DNA encoding SEQ ID NO 2
atggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 393 aa, Artificial sequence
  This is a fusion protein sequence containing Interferon-beta and a portion of human immunoglobulin heavy chain sequence
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1179 bp, Artificial sequence
  DNA encoding SEQ ID NO 1
atgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2010/0256337 A1
(43) Pub. Date: Oct. 7, 2010

(54) METHOD FOR PURIFYING AN FC-CONTAINING PROTEIN

(75) Inventor: Alex Eon-Duval, Vevey (CH)

Correspondence Address: SALIWANCHIK LLOYD & SALIWANCHIK;A PROFESSIONAL ASSOCIATION, PO Box 142950, GAINESVILLE, FL, 32614, US

(73) Assignee: MERCK SERONO SA, COINSINS, VAUD (CH)

(21) Appl. No.: 12/738,515

(22) PCT Filed: Oct. 21, 2008
(86) PCT No.: PCT/EP08/64210

§ 371 Date: Apr. 16, 2010

Related U.S. Application Data
(63) Continuation-in-part of application No. 61/007,223, filed on Dec. 11, 2007.

(30) Foreign Application Priority Data
Oct. 22, 2007 (EP) 07118983.1

(51) Int. Cl. C07K 16/00

(52) U.S. Cl. 530/387.3

(57) ABSTRACT

The invention relates to a method for the purification of an Fc-containing protein via blue dye affinity chromatography, in particular for the reduction of the amount of free Fc-moieties in an Fc-containing protein preparation.

### FIELD OF THE INVENTION

The present invention is in the field of protein purification. More specifically, it relates to the purification of an Fc-containing protein via blue dye affinity chromatography, in particular for the reduction of the amount of free Fc-moieties in an Fc-containing protein preparation.

### BACKGROUND OF THE INVENTION

Proteins have become commercially important as drugs that are generally called “biologicals”. One of the greatest challenges is the development of cost effective and efficient processes for purification of proteins on a commercial scale. While many methods are now available for large-scale preparation of proteins, crude products, such as cell culture supernatants, contain not only the desired product but also impurities, which are difficult to separate from the desired product. Although cell culture supernatants of cells expressing recombinant protein products may contain fewer impurities if the cells are grown in serum-free medium, the host cell proteins (HCPs) still remain to be eliminated during the purification process. Additionally, the health authorities request high standards of purity for proteins intended for human administration.

A number of chromatographic methods are known that are widely used for protein purification. Methods such as affinity chromatography and the like have been widely used.

Blue dye affinity chromatography is based on a dye-ligand, Cibacron Blue, which is bound to a matrix (e.g. sepharose or agarose). In the Blue Sepharose resin, the ligand Cibacron Blue F3G-A, is covalently coupled to sepharose™ through chlorotriazine ring Vlatakis G et al., 1987). Blue Sepharose has been mainly used for the purification of interferon beta (Knight E Jr and Fahey, 1981) and albumin. Examples of commercially available blue dye affinity matrices include Blue Sepharose 6FF resin (GE Healthcare), Blue Sepharose CL-6B (GE Healthcare), Blue Trisacryl M (Pall/BioSepra), Affi-Gel Blue (Bio-Rad), Econo-Pac blue cartridges (Bio-Rad), SwellGel Blue (Pierce), Toyopearl AF-Blue (Tosoh Bioscience) or Cibacron Blue F3GA (Polysciences Inc.).

Ion exchange chromatography systems are used for separation of proteins primarily on the basis of differences in charge.

Affinity chromatography is based on the affinity of a protein of interest to another protein that is immobilized to a chromatography resin. Examples for such immobilized ligands are the bacterial cell wall proteins Protein A and Protein G, having specificity to the Fc portion of certain immunoglobulins (Igs). Although both Protein A and Protein G have a strong affinity for IgG antibodies, they have varying affinities to other immunoglobulin classes and isotypes as well.

Protein A, Protein G, and Protein L affinity chromatography are widely used for isolation and purification of antibodies.

Since the binding sites for Protein A and Protein G reside in the Fc region of an immunoglobulin, Protein A and Protein G (or Protein NG) affinity chromatography also allows purification of so-called Fc-fusion proteins. Protein L binds to Ig light chains and can thus be used for the purification of light chain containing antibodies.

Antibodies, or immunoglobulins (Igs) consist of light chains and heavy chains linked together by disulphide bonds. The first domain located at the amino terminus of each chain is variable in amino acid sequence, providing the vast spectrum of antibody binding specificities. These domains are known as variable heavy (VH) and variable light (VL) regions. The other domains of each chain are relatively invariant in amino acid sequence and are known as constant heavy (CH) and constant light (CL) regions.

The major classes of antibodies are IgA, IgD, IgE, IgG and IgM; and these classes may be further divided into subclasses (isotypes). For example, the IgG class has four subclasses, namely, IgG₁, IgG₂, IgG₃, and IgG₄.

The differences between antibody classes are derived from differences in the heavy chain constant regions, containing between 1 and 4 constant domains (CH1-CH4), depending on the immunoglobulin class. A so-called hinge region is located between the CH1 and CH2 domains. The hinge region is particularly sensitive to proteolytic cleavage; such proteolysis yields two or three fragments depending on the precise site of cleavage. The part of the heavy chain constant region containing the CH2 and CH3 domains is also called the “Fc” part of the immunoglobulin. Antibodies are thus Fc-containing proteins. Another type of Fc-containing proteins are the so-called Fc-fusion proteins.

Several antibodies that are used as therapeutic proteins are known. Examples for recombinant antibodies on the market are for instance: Abciximab, Rituximab, Basiliximab, Daclizumab, Palivizumab, Infliximab, Trastuzumab, Alemtuzumab, Adalimumab, Cetuximab, Efalizumab, Ibritumomab, Bevacizumab, or Omalizumab.

Another type of Fc-containing proteins are the so-called Fc-fusion proteins. Fc-fusion proteins are chimeric proteins consisting of the effector region of a protein, such as the Fab region of an antibody or the binding region of a receptor, fused to the Fc region of an immunoglobulin that is frequently an immunoglobulin G (IgG). Fc-fusion proteins are widely used as therapeutics as they offer advantages conferred by the Fc region, such as:
The possibility of purification using protein A or protein G affinity chromatography with affinities which vary according to the IgG isotype. Human IgG₁, IgG₂ and IgG₄ bind strongly to Protein A and all human IgGs including IgG₃ bind strongly to Protein G;
An increased half-life in the circulatory system, since the Fc region binds to the salvage receptor FcRn which protects from lysosomal degradation;
Depending on the medical use of the Fc-fusion protein, the Fc effector functions may be desirable. Such effector functions include antibody-dependent cellular cytotoxicity (ADCC) through interactions with Fc receptors (FcγRs) and complement-dependent cytotoxicity (CDC) by binding to the complement component 1q (C1q). IgG isoforms exert different levels of effector functions. Human IgG₁ and IgG₃ have strong ADCC and CDC effects while human IgG₂ exerts weak ADCC and CDC effects. Human IgG₄ displays weak ADCC and no CDC effects.

Serum half-life and effector functions can be modulated by engineering the Fc region to increase or reduce its binding to FcRn, FcγRs and C1q respectively, depending on the therapeutic use intended for the Fc-fusion protein.

In ADCC, the Fc region of an antibody binds to Fc receptors (FcγRs) on the surface of immune effector cells such as natural killers and macrophages, leading to the phagocytosis or lysis of the targeted cells.

In CDC, the antibodies kill the targeted cells by triggering the complement cascade at the cell surface. IgG isoforms exert different levels of effector functions increasing in the order of IgG4<IgG2<IgG1≦IgG3. Human IgG1 displays high ADCC and CDC, and is the most suitable for therapeutic use against pathogens and cancer cells.

Under certain circumstances, for example when depletion of the target cell is undesirable, abrogating effector functions is required. On the contrary, in the case of antibodies intended for oncology use, increasing effector functions may improve their therapeutic activity (Carter et al., 2006).

Modifying effector functions can be achieved by engineering the Fc region to either improve or reduce binding of FcγRs or the complement factors.

The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Two regions of the CH2 domain are critical for FcγRs and complement C1q binding, and have unique sequences in IgG2 and IgG4. For instance, substitution of IgG2 residues at positions 233-236, according to EU index position as defined by Kabat et al. (Kabat, E. A., Wu, T. T., Perry, H. M., Gottesman, K. S., and Foeller, C. (1991), into human IgG1 greatly reduced ADCC and CDC (Armour et al., 1999 and Shields et al., 2001).

Numerous mutations have been made in the CH2 domain of IgG and their effect on ADCC and CDC was tested in vitro (Shields et al., 2001, Idusogie et al., 2001 and 2000, Steurer et al., 1995). In particular, a mutation to alanine at E333 was reported to increase both ADCC and CDC (Idusogie et al., 2001 and 2000).

Increasing the serum half-life of a therapeutic antibody is another way to improve its efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton et al., 2004 and Vaccaro et al., 2005).

The following Table 1 summarizes some known mutations of the IgG Fc-region (taken from Invivogen's website).

| Engineered | IgG | | | | |
|---|---|---|---|---|---|
| Fc | Isotype | Mutations | Properties | Potential Benefits | Applications |
| hIgG1e1 | human | T250Q/M428L | Increased | Improved localization | Vaccination; |
| | IgG1 | | plasma half- | to target; increased | therapeutic |
| | | | life | efficacy; reduced | use |
| | | | | dose or frequency of | |
| | | | | administration | |
| hIgG1e2 | human | M252Y/S254T/T256E + | Increased | Improved localization | Vaccination; |
| | IgG1 | H433K/N434F | plasma half- | to target; increased | therapeutic |
| | | | life | efficacy; reduced | us |
| | | | | dose or frequency of | |
| | | | | administration | |
| hIgG1e3 | human | E233P/L234V/L235A/ | Reduced | Reduced adverse | Therapeutic |
| | IgG1 | ΔG236 + A327G/ | ADCC and | events | use without |
| | | A330S/P331S | CDC | | cell depletion |
| hIgG1e4 | human | E333A | Increased | Increased efficacy | Therapeutic |
| | IgG1 | | ADCC and | | use with cell |
| | | | CDC | | depletion |
| hIgG2e1 | human | K322A | Reduced | Reduced adverse | Vaccination; |
| | IgG2 | | CDC | events | therapeutic |
| | | | | | use |

In one class of Fc-fusion proteins having therapeutic utility, Fc-regions have been fused to extracellular domains of certain receptors belonging to the tumor necrosis factor receptor (TNF-R) superfamily (Locksley et al., 2001, Bodmer et al., 2002, Bossen et al., 2006). A hallmark of the members of the TNFR family is the presence of cysteine-rich pseudo-repeats in the extracellular domain, as described e.g. by Naismith and Sprang, 1998.

The two TNF receptors, p55 (TNFR1) and p75 TNFR (TNFR2) are examples of such members of the TNFR superfamily. Etanercept is an Fc-fusion protein containing the soluble part of the p75 TNFR (e.g. WO 91/03553, WO 94/06476). Under the trade name Enbrel®, it is marketed for treatment of Endometriosis, Hepatitis C virus infection, HIV infection, Psoriatic arthritis, Psoriasis, Rheumatoid arthritis, Asthma, Ankylosing spondylitis, Cardiac failure, Graft versus host disease, Pulmonary fibrosis, Crohns disease. Lenercept is a fusion protein containing extracellular components of human p55 TNF receptor and the Fc portion of human IgG, and is intended for the potential treatment of severe sepsis and multiple sclerosis.

OX40 is also a member of the TNFR superfamily. OX40-IgG1 and OX40-hIG4mut fusion proteins have been prepared for treatment of inflammatory and autoimmune diseases such as Crohn's Disease.

An Fc-fusion protein of the BAFF-R, also called BR3, designated BR3-Fc, is a soluble decoy receptor from a series of inhibitors of BAFF (B-cell activating factor of the TNF family), is being developed for the potential treatment of autoimmune diseases such as rheumatoid arthritis (RA) and systemic lupus erythematosus (SLE).

BCMA is a further receptor belonging to the TNFR superfamily. A BCMA-Ig fusion protein has been described to inhibit autoimmune disease (Melchers, 2003).

Another receptor of the TNF-R superfamily is TACI, the transmembrane activator and CAML-interactor (von Bülow and Bram, 1997; U.S. Pat. No. 5,969,102, Gross et al., 2000), which has an extracellular domain containing two cysteine-rich pseudo-repeats. TACI binds two members of the tumor necrosis factor (TNF) ligand family. One ligand is designated BLyS, BAFF, neutrokine-a, TALL-1, zTNF4, or THANK (Moore et al., 1999). The other ligand has been designated as APRIL, TNRF death ligand-1 or ZTNF2 (Hahne et al., 1998).

Fusion proteins containing soluble forms of the TACI receptor fused to an IgG Fc region are known and were designated TACI-Fc (WO 00/40716, WO 02/094852). TACI-Fc inhibits the binding of BLyS and APRIL to B-cells (Xia et al., 2000). It is being developed for the treatment of autoimmune diseases, including systemic lupus erythematosus (SLE), rheumatoid arthritis (RA) and hematological malignancies, as well as for treatment of multiple sclerosis (MS). In addition to this, a TACI-Fc, designated atacicept, is being developed in multiple myeloma (MM) (Novak et al., 2004) and non-Hodgkin's lymphoma (NHL), chronic lymphocytic leukemia (CLL) and Waldenstrom's macroglobulemia (WM).

Another example of Fc-fusion protein consists of an Fc region linked to a single interferon beta protein. Interferon beta (interferon-β or IFN-β) is a naturally occurring soluble glycoprotein belonging to the class of cytokines. Interferons (IFNs) have a wide range of biological activities, such as anti-viral, anti-proliferative and immunomodulatory properties. Interferon beta is used as a therapeutic protein drug, a so-called biological, in a number of diseases, such as e.g. multiple sclerosis, cancer, or viral diseases such as e.g. SARS or hepatitis C virus infections.

Fusion proteins containing IFN-β as a biologically active molecule fused to an IgG Fc region are described in WO2005/001025.

Given the therapeutic utility of Fc-containing proteins, particularly antibodies and Fc-fusion proteins, there is a need for significant amounts of highly purified protein that is adequate for human administration.

### SUMMARY OF THE INVENTION

One of the problems that may be encountered during production of Fc-containing proteins is the presence of “free Fc-moieties”, i.e. polypeptide fragments derived from the Fc-containing protein, which are neither fused to antibody variable regions nor to other specific proteins or domains normally present in the Fc-fusion protein. The free Fc-moieties may result from the expression of a heterodimeric fusion protein, e.g. a single protein fused to an Fc region, or from proteolytic cleavage of the protein of interest.

The present invention addresses this problem. It is based on the development of a purification method for a fluid, composition or preparation of an Fc-containing protein, by which the amount of free Fc-moieties that may be present as an impurity can be reduced.

Therefore the invention relates to a method for reducing the concentration of free Fc-moieties in a fluid comprising an Fc-containing protein, the method comprising subjecting said fluid to blue sepharose chromatography and eliminating the free Fc-moieties by washing the resin at a pH ranging from about 4.0 to 6.0.

In a second aspect, the invention relates to the use of blue sepharose chromatography for the reduction of free Fc in an Fc-containing protein preparation.

In a third aspect, the invention relates to a purified Fc-containing protein, comprising less than about 5% or less than about 2% or less than about 1% or less than about 0.5% or less than about 0.2% or less than about 0.1% of free Fc-moieties.

### BRIEF DESCRIPTION OF THE DRAWINGS

FIG. 1 shows the chromatographic profile of the Blue Sepharose chromatography described in Example 1. (1) Peak 1; (2) Peak 2.

FIG. 2 shows a silver stained SDS-PAGE of different fractions stemming from the blue sepharose chromatography described in Example 1 under non-reducing (A) and reducing (B) conditions.

Lane 1: Molecular weight markers

Lane 2: purified TACI-Fc

Lane 3: Load

Lane 4: Peak 1

Lane 5: Peak 2

Lane 6: Peaks 1+2

Lane 7: Molecular weight markers

FIG. 3 shows photographs of a western immunoblotting analysis using anti-TACI antibodies and Anti-Fc antibodies of fractions stemming from the blue sepharose chromatography described in Example 1 (lanes 2 to 5 and 9 to 12 only; lanes 6 to 8 and 13 to 15 represent fractions stemming from a wash under different conditions):

FIG. 3A Anti-TACI detection under non-reducing (Lanes 2 to 5) and reducing (Lanes 9 to 12) conditions

FIG. 3B Anti-Fc detection under non-reducing (Lanes 2 to 5) and reducing (Lanes 9 to 12) conditions.

Lanes 2 and 9: purified TACI-Fc

Lane 3, 10: load

Lane 4, 11: Peak 1

Lane 5, 12: Peak 2

FIG. 4 MALDI-MS analysis spectrum of the load (FIG. 4.A), peak 1 (FIG. 4.B.), and peak 2 (FIG. 4.C) fractions from Example 1 where peak (a) represents free Fc, peak (b) hybrid TACI-Fc/Fc and peak (c) intact TACI-Fc.

FIG. 5 Shows the chromatographic profile of the blue sepharose chromatography described in Example 3. (i) OD at 280 nm (mAU), (ii) KCl concentration, (iii) Conductivity, (iv) pH. 1-Load, 2-Wash 1, 3-Wash 2, 4-Wash 3, 5-Wash 4, 6-Wash 5, 7-Wash 6, 8-Elution, 9-Regeneration.

### BRIEF DESCRIPTION OF THE SEQUENCE LISTING

SEQ ID NO: 1 is a Cysteine fingerprint sequence common to members of the TNFR superfamily;

SEQ ID NO: 2 is a preferred Fc-fusion protein of the invention, comprising sequences derived from the extracellular portion of TACI and a human IgG1 Fc portion (e.g. described in WO 02/094852);

SEQ ID NO: 3 is a polynucleotide coding for a polypeptide of SEQ ID NO: 2;

SEQ ID NO: 4 is a preferred IFNβ-Fc amino acid sequence. Amino acids 1 to 166 represent the mature human interferon beta and amino acids 167 to 393 represent a portion of a mutated human immunoglobulin gamma heavy chain sequence.

SEQ ID NO: 5 is a polynucleotide coding for a polypeptide of SEQ ID NO: 4.

### DETAILED DESCRIPTION OF THE INVENTION

The present invention is based on the finding that blue dye affinity chromatography can provide a convenient and simple way to efficiently reduce the amount or extent of free Fc-moieties that may be present in a fluid or composition of an Fc-containing protein increasing thereby the purity of the Fc-containing protein. The free Fc-moieties may result from the expression of a heterodimeric fusion protein, e.g. a single protein fused to an Fc region, or from proteolytic cleavage of the protein of interest.

The invention therefore relates to a method for purifying an Fc-containing protein from free Fc-moieties present in a fluid comprising said Fc-containing protein, the method comprising the steps of:
(a) loading said fluid on a blue dye affinity chromatography resin;
(b) washing the resin with a buffer having a pH of about 4.0 to about 6.0 thereby eliminating the free Fc-moieties from the resin; and
(c) eluting the Fc-containing protein from the resin.

The fluid comprising the Fc-containing protein may be any composition or preparation, such as e.g. a body fluid derived from a human or animal, or a fluid derived from a cell culture, such as e.g. a cell culture supernatant or cell culture harvest. It may also be a fluid derived from another purification step, such as e.g. the eluate or flow-through from a capture step or any other suitable purification step preceding the blue sepharose chromatography such as the eluate of protein A chromatography.

The fluid may preferably be cell culture material, e.g. solubilised cells, more preferably cell culture supernatant. The term “cell culture supernatant”, as used herein, refers to a medium in which cells are cultured and into which proteins are secreted provided they contain appropriate cellular signals, so-called signal peptides. It is preferred that the Fc-containing protein expressing cells are cultured under serum-free culture conditions. Thus, preferably, the cell culture supernatant is devoid of animal derived components. Most preferably, the cell culture medium is a chemically defined medium.

Preferably, the protein purified according to the invention is a Fc-containing protein such as, e.g. an antibody, more preferably a human, humanized or chimeric antibody comprising human constant regions, preferably an IgG1 antibody, it can also preferably be an Fc-fusion protein. Fc-containing proteins are chimeric proteins consisting of the effector region of a protein, such as e.g. the Fab region of an antibody or the binding region of a receptor, fused to the Fc region of an immunoglobulin that is frequently an immunoglobulin G (IgG).

Herein, an Fc region may be referred to as an Fc fragment or Fc domain. Herein, the terms “Fc region”, “Fc fragment” or “Fc domain” are interchangeable and should be construed as having the same meaning.

The term “Fc-containing protein”, as used herein, refers to any protein having at least one immunoglobulin constant domain, preferably human constant region, selected from the CH1, hinge, CH2, CH3, CH4 domain, or any combination thereof, and preferably a hinge, CH2 and CH3 domain. The immunoglobulin constant domain may be derived from any of IgG, IgA, IgE, IgM, or combination or isotype thereof. Preferably, it is IgG, such as e.g. IgG₁, IgG₂, IgG₃ or IgG₄. More preferably, it is IgG₁.

An Fc-containing protein, in accordance with the present invention, may thus be e.g. an antibody or an Fc-fusion protein, or variants thereof, such as fragments, muteins or functional derivatives of antibodies or Fc-fusion proteins.

The Fc-containing protein of the invention may be a monomer, dimer or multimer. The Fc-containing protein may also be a “pseudo-dimer” (sometimes called “monomer”), containing a dimeric Fc-moiety (e.g. a dimer of two disulfide-bridged hinge-CH2-CH3 constructs), of which only one is fused to a further moiety such as an immunoglobulin variable domain, a ligand binding fragment of a receptor, or any other protein. An example for such a pseudo-dimer is an Fc-fusion protein having Interferon-β fused to one of the two IgG hinge-CH2-CH3 constructs such as e.g. the one described in WO 2005/001025.

The Fc-containing protein may also be a heterodimer, containing two different non-immunoglobulin portions or immunoglobulin variable domains, or a homodimer, containing two copies of a single non-immunoglobulin portion or immunoglobulin variable domain.

Preferably, the Fc-containing protein is a dimer. It is also preferred that the Fc-containing protein of the invention is a homo-dimer.

In accordance with the present invention, the Fc-moiety of the Fc-containing protein may also be modified in order to modulate effector functions.

For instance, the following Fc mutations, according to EU index positions (Kabat et al., 1991), can be introduced if the Fc-moiety is derived from IgG1:

T250Q/M428L

M252Y/S254T/T256E+H433K/N434F

E233P/L234V/L235A/ΔG236+A327G/A330S/P331S

E333A; K322A.

Further Fc mutations may e.g. be the substitutions at EU index positions selected from 330, 331 234, or 235, or combinations thereof. An amino acid substitution at EU index position 297 located in the CH2 domain may also be introduced into the Fc-moiety in the context of the present invention, eliminating a potential site of N-linked carbohydrate attachment. Furthermore, the cysteine residue at EU index position 220 may also be replaced with a serine residue, eliminating the cysteine residue that normally forms disulfide bonds with the immunoglobulin light chain constant region.

In a preferred embodiment, the Fc-containing protein comprises an immunoglobulin variable region, e.g. one or more heavy chain variable domains and/or one or more light chain variable domains. Preferably, the antibody contains one or two heavy chain variable domains. More preferably, the antibody additionally contains one or two light chain constant and/or variable domains.

In a preferred embodiment of the invention, the Fc-containing protein that can be purified according to the invention is an antibody. Preferably, said antibody is a monoclonal antibody. The antibody may be a chimeric antibody, a humanized antibody or a human antibody. The antibody may either be produced in a host cell transfected with one, two or more polynucleotides coding for the antibody or produced from a hybridoma.

As used herein, the term “antibody” refers to a Fc-containing protein wherein the therapeutic moiety comprises at least one variable domain of an immunoglobulin (Ig). Preferred immunoglobulins are mammalian immunoglobulins. More preferred immunoglobulins are camelid immunoglobulins. Even more preferred immunoglobulins are rodent immunoglobulins, in particular from rat or mouse. Most preferred immunoglobulins are primate immunoglobulins, in particular human immunoglobulins.

An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions retain the binding specificity to the antigen and can be further subdivided into regions of hypervariability, termed complementarity determining regions (CDR) The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, FR4. The variable regions of the heavy and light chains contain a binding domain that interacts with an antigen.

Examples of antibodies that can be purified in accordance with the present invention are antibodies directed against a protein selected from the group consisting of CD3 (e.g. OKT3, NI-0401), CD11a (e.g. efalizumab), CD4 (e.g. zanolimumab, TNX-355), CD20 (e.g. ibritumomab tiuxetan, rituximab, tositumomab, ocrelizumab, ofatumumab, IMMU-106, TRU-015, AME-133, GA-101), CD23 (e.g. lumiliximab), CD22 (e.g. epratuzumab), CD25 (e.g. basiliximab, daclizumab), the epidermal growth factor receptor (EGFR) (e.g. panitumumab, cetuximab, zalutumumab, MDX-214), CD30 (e.g MDX-060), the cell surface glycoprotein CD52 (e.g. alemtuzumab), CD80 (e.g. galiximab), the platelet GPIIb/IIIa receptor (e.g. abciximab), TNF alpha (e.g. infliximab, adalimumab, golimumab), the interleukin-6 receptor (e.g. tocilizumab), carcinoembryonic antigen (CEA) (e.g. 99mTc-besilesomab), alpha-4/beta-integrin (VLA4) (e.g. natalizumab), alpha-5/beta-1 integrin (VLA5) (e.g. volociximab), VEGF (e.g. bevacizumab, ranibizumab), immunoglobulin E (IgE) (e.g. omalizumab), HER-2/neu (e.g. trastuzumab), the prostate specific membrane antigen (PSMA) (e.g. 111In-capromab pendetide, MDX-070), CD33 (e.g. gemtuzumab ozogamicin), GM-CSF (e.g. KB002, MT203), GM-CSF receptor (e.g. CAM-3001), EpCAM (e.g. adecatumumab), IFN-gamma (e.g. NI-0501), IFN-alpha (e.g. MEDI-545/MDX-1103), RANKL (e.g. denosumab), hepatocyte growth factor (e.g. AMG 102), IL-15 (e.g. AMG 714), TRAIL (e.g. AMG 655), insulin-like growth factor receptor (e.g. AMG 479, R1507), IL-4 and IL13 (e.g. AMG 317), BAFF/BLyS receptor 3 (BR3) (e.g. CB1), CTLA-4 (e.g. ipilimumab).

Preferably, the antibodies that can be purified in accordance with the present invention are antibodies directed against a protein selected from the group consisting of CD3, CD4, CD11a, CD25, IFN-gamma, EpCAM, TACI.

Most preferably, said antibody is selected from the group consisting of an anti-CD4 antibody (see e.g. WO 97/13852), an anti-CD11a antibody (see e.g. WO 98/23761) and an anti-CD25 antibody (see e.g. WO 2004/045512).

Antibodies directed against TNF, Blys, or Interferon-y are further examples of therapeutically interesting antibodies.

Fc-fusion proteins are also Fc-containing proteins that are preferably subjected to the method of the invention.

The term “Fc-fusion protein”, as used herein, is meant to encompass proteins, in particular therapeutic proteins, comprising an immunoglobulin-derived moiety, which will be called herein the “Fc-moiety”, and a moiety derived from a second, non-immunoglobulin protein, which will be called herein the “therapeutic moiety”, irrespective of whether or not treatment of disease is intended.

Therapeutic Fc-fusion proteins, i.e. Fc-fusion proteins intended for treatment or prevention of disease of an animal or preferably for human treatment or administration, are especially suitable to be purified in accordance with the invention.

Any Fc-fusion protein may be purified in accordance with the present invention, such as e.g. an Interferon-β-containing fusion protein. Preferably, the method of the invention is for purifying an Fc-fusion protein comprising a ligand binding fragment, such as all or part of an extracellular domain, of a member of the tumor necrosis factor receptor (TNFR) superfamily.

The therapeutic moiety of an Fc-fusion protein may e.g. be or be derived from EPO, TPO, Growth Hormone, Interferon-alpha, Interferon-beta, Interferon-gamma, PDGF-beta, VEGF, IL-1alpha, IL-1beta, IL-2, IL-4, IL-5, IL-8, IL-10, IL-12, IL-18, IL-18 binding protein, TGF-beta, TNF-alpha, or TNF-beta.

The therapeutic moiety of an Fc-fusion protein may also be derived from a receptor, e.g a transmembrane receptor, preferably be or be derived from the extracellular domain of a receptor, and in particular a ligand binding fragment of the extracellular part or domain of a given receptor. Examples for therapeutically interesting receptors are CD2, CD3, CD4, CD8, CD11a, CD11b, CD14, CD18, CD20, CD22, CD23, CD25, CD33, CD40, CD44, CD52, CD80, CD86, CD147, CD164, IL-2 receptor, IL-4 receptor, IL-6 receptor, IL-12 receptor, IL-18 receptor subunits (IL-18R-alpha, IL-18R-beta), EGF receptor, VEGF receptor, integrin alpha 4 10 beta 7, the integrin VLA4, B2 integrins, TRAIL receptors 1, 2, 3, and 4, RANK, RANK ligand, epithelial cell adhesion molecule (EpCAM), intercellular adhesion molecule-3 (ICAM-3), CTLA4 (which is a cytotoxic T lymphocyte- associated antigen), Fc-gamma-I, II or III receptor, HLA-DR 10 beta, HLA-DR antigen, L-selectin.

It is highly preferred that the therapeutic moiety is derived from a receptor belonging to the TNFR superfamily. The therapeutic moiety may e.g. be or be derived from the extracellular domain of TNFR1 (p55), TNFR2 (p75), OX40, Osteoprotegerin, CD27, CD30, CD40, RANK, DR3, Fas ligand, TRAIL-R1, TRAIL-R2, TRAIL-R3, TAIL-R4, NGFR, AITR, BAFFR, BCMA, TACI.

In accordance with the present invention, the therapeutic moiety derived from a member of the TNFR superfamily preferably comprises or consists of all or part of the extracellular domain of the member of the TNFR, and more preferably comprises a ligand binding fragment of such a member of the TNFR.

The following Table 2 lists members of the TNFR superfamily from which a therapeutic moiety in accordance with the present invention may be derived, and their respective ligands. A “ligand binding fragment” of a member of the TNFR family can easily be determined by the person skilled in the art, e.g. in a simple in vitro assay measuring binding between protein fragment of a given receptor and the respective ligand. Such an assay can e.g. be a simple in vitro RIA- or ELISA-type sandwich assay wherein one of the proteins, e.g. the receptor fragment, is immobilized to a carrier (e.g. an ELISA plate) and is incubated, following appropriate blocking of the protein binding sites on the carrier, with the second protein, e.g. the ligand. After incubation, ligand binding is detected e.g. by way of radioactive labelling of the ligand and determination of the bound radioactivity, after appropriate washing, in a scintillation counter. Binding of the ligand can also be determined with a labelled antibody, or a first ligand-specific antibody and a second, labelled antibody directed against the constant part of the first antibody. Ligand binding can thus be easily determined, depending of the label used, e.g. in a colour reaction.

Preferably, the method of the present invention is for purifying an Fc-fusion protein comprising a therapeutic moiety derived from a member of the TNFR superfamily selected from those listed in Table 1.

TABLE 2
The TNFR superfamily (according to Locksley et al.,
2001 and Bossen et al., 2006)

| Member of | |
|---|---|
| TNFR superfamily | Ligand |
| NGFR | NGF |
| EDAR | EDA-A1 |
| XEDAR | EDA-A2 |
| CD40 | CD40L |
| Fas | FasL |
| Ox40 | OX40L |
| AITR | AITRL |
| GITR | GITRL |
| CD30 | CD30L |
| CD40 | CD40L |
| HveA | LIGHT, LT-alpha |
| 4-1BB | 4-1BBL |
| TNFR2 | TNF-alpha, LT-alpha, LT-alpha-beta |
| LT-betaR | LIGHT, LT-alpha, LT-alpha-beta |
| DR3 | TL1A |
| CD27 | CD27L |
| TNFR1 | TNF-alpha, LT-alpha, LT-alpha-beta |
| LTBR | LT-beta |
| RANK | RANKL |
| TACI | BlyS, APRIL |
| BCMA | BlyS, APRIL |
| BAFF-R | BAFF (= BlyS) |
| TRAILR1 | TRAIL |
| TRAILR2 | TRAIL |
| TRAILR3 | TRAIL |
| TRAILR4 | TRAIL |
| Fn14 | TWEAK |
| OPG | RANKL, TRAIL |
| DR4 | TRAIL |
| DR5 | TRAIL |
| DcR1 | TRAIL |
| DcR2 | TRAIL |
| DcR3 | FasL, LIGHT, TL1A |

In a preferred embodiment, the Fc-fusion protein comprises a therapeutic moiety selected from an extracellular domain of TNFR1, TNFR2, or a TNF binding fragment thereof.

In a further preferred embodiment, the Fc-fusion protein comprises a therapeutic moiety selected from an extracellular domain of BAFF-R, BCMA, or TACI, or a fragment thereof binding at least one of Blys or APRIL.

An assay for testing the capability of binding to Blys or APRIL is described e.g. in Hymowitz et al., 2005.

In yet a further preferred embodiment, the therapeutic moiety of an Fc-fusion protein comprises the Cysteine rich pseudo-repeat of SEQ ID NO: 1.

It is further preferred that the therapeutic moiety is derived from TACI. TACI is preferably human TACI. More preferably, the therapeutic moiety comprises a soluble portion of TACI, preferably derived from the extracellular domain of TACI (the amino acid sequence of human full-length TACI receptor corresponds to SwissProt entry O14836). A highly preferred Fc-fusion protein to be purified in accordance with the present invention comprises or consists of SEQ ID NO: 2 or encoded by the polynucleotide of SEQ ID NO: 3.

Hence, it is highly preferred that the Fc-fusion protein comprises a polypeptide selected from
(a) SEQ ID NO: 2;
(b) a polypeptide encoded by a polynucleotide hybridizing to the complement of SEQ ID NO: 3 under highly stringent conditions; and
(c) a mutein of (a) having at least 80% or 85% or 90% or 95% sequence identity to the polypeptide of (a);
wherein the polypeptide binds to at least one of Blys or APRIL.

wherein the polypeptide binds to at least one of Blys or APRIL.

Therapeutic Fc-fusion proteins, i.e. Fc-fusion proteins intended for treatment or prevention of disease of an animal or preferably for human treatment or administration, are especially suitable for use in the frame of the invention, to be purified in accordance with the invention.

Most preferably, said Fc-fusion protein comprises either a fragment of the TACI receptor (see e.g. WO 02/094852) or a fragment of IFN-β (see e.g. WO 2005/001025).

According to the present invention, a fusion protein comprising IFN-β preferably comprises a polypeptide selected from
(a) SEQ ID NO: 4;
(b) a polypeptide encoded by a polynucleotide hybridizing to the complement of SEQ ID NO: 5 under highly stringent conditions; and
(c) a mutein of (a) having at least 80% or 85% or 90% or 95% sequence identity to the polypeptide of (a);

In accordance with the present invention, the Fc-containing protein is subjected to blue sepharose chromatography in order to reduce, decrease, or eliminate free Fc-moieties, preferably at least to less than 5%, 2%, 1%, 0.5%, 0.2% or 0.1% of the Fc-containing protein.

The term “free Fc moieties”, “free Fc moiety”, or simply “free Fc”, as used herein, is meant to encompass any part of the Fc-containing protein to be purified in accordance with the present invention, which is derived from the immunoglobulin constant domain or domains without comprising complete further domains. Thus, if the Fc-containing protein comprises immunoglobulin variable domains, free Fc does not contain significant portions of the variable domains. If the Fc-containing protein is an Fc-fusion protein, free Fc does not contain significant portions of the therapeutic moiety of the Fc-fusion protein. Free Fc may e.g. contain dimers of the IgG hinge, CH2 and CH3 domains, which are not linked to significant portions of a therapeutic moiety or immunoglobulin variable domains, such as e.g. the Fc part that is generated by papain cleavage.

Monomers derived from the Fc-moiety may also be contained in the free Fc fraction. It is understood that free Fc may still contain a number of amino acid residues from the therapeutic moiety or the Ig variable domains, such as e.g. one to fifty or one to twenty, or one to ten, or one to five amino acids, or one single amino acid, belonging to the therapeutic moiety or variable domain, still fused to the Fc-moiety.

In accordance with the present invention, the blue dye affinity chromatography may be carried out on any suitable resin, and preferably the resin comprises Cibacron Blue F3G-A ligand. Preferably, the blue dye affinity chromatography is carried out on Blue Sepharose resin. A resin commercially available under the name Blue Sepharose 6FF resin (GE Healthcare) is an example of an affinity resin that is particularly suitable for step (a) of the present method. The technical features of Blue Sepharose FF are as follows:

TECHNICAL SPECIFICATIONS

| | |
|---|---|
| Ligand | Cibacron Blue F3G-A |
| Ligand coupling | Triazine coupling |
| method | |
| Binding capacity | >18 mg human serum albumin/ml |
| | medium |
| Ligand density | ≈7 μmol Cibacron Blue/ml medium |
| Matrix | Highly cross-linked agarose, 6% |
| Average particle size | 90 μm |
| pH stability | 4-12 (long term), 3-13 (short term) |
| Chemical stability | 40° C. for 7 days in: 70% ethanol, 6 M |
| | guanidine hydrochloride, 8 M urea |

Other suitable commercially avaialble blue dye affinity columns are selected from Blue Sepharose CL-6B (GE Healthcare), Blue Trisacryl M (Pall/BioSepra), Affi-Gel Blue (Bio-Rad), Econo-Pac blue cartridges (Bio-Rad), SwellGel Blue (Pierce), Toyopearl AF-Blue (Tosoh Bioscience) or Cibacron Blue F3GA (Polysciences Inc.).

In step (a) of the purification method of the invention, before loading the fluid comprising an Fc-containing protein on the blue dye resin, the fluid is preferably either adjusted to a pH of less than 6 preferably about 5 and if necessary diluted with water to a conductivity of less than about 20 mS/cm at about pH 5. This allows binding of the Fc-containing protein to the blue dye resin.

The pH of less than 6 may e.g. be at about 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1 or at about 2.0.

In step (b) of the method of the invention, the free Fc-moieties are washed from the blue dye resin with a buffer having a pH of about 4.0 to about 6.0 thereby. The pH may e.g. be at about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or at about 6.0.

In step (b), the free Fc-moieties are washed out from the blue sepharose resin using any suitable salt. A salt selected from potassium chloride or sodium chloride is preferred. An increasing salt gradient ranging from about 0 to about 0.5 M potassium chloride at pH 5 is preferred.

In a preferred embodiment, the free Fc-moieties are eluted from the blue sepharose resin in step (b) with an increasing salt gradient ranging from about 0 to about 5M KCl. The increasing salt gradient can e.g. range from about 0 to about 500, 50 to 500, 100 to 500, 150 to 500, mM KCl at pHS.

In a further preferred embodiment, the Fc moieties are washed from the blue sepharose column with an isocratic salt concentration ranging from 200 to 300 mM KCl at pHS. The isocratic salt concentration can e.g. be 200, 210, 220, 230, 240, 250 , 260, 270, 280, 290 mM KCl at pHS. It is preferably 240 mM KCl at pHS.

In a further preferred embodiment, step (b) is carried out in a buffer comprising about 10 to about 100, preferably 15 to 90, more preferably 20 to 80 mM sodium acetate. The buffer may e.g. comprise 10, 15, 20, 25, 30, 35, 40, 45, 50, 55, 60, 65, 70, 75, 80, 85, 90, 95, 100 mM sodium acetate.

In a preferred embodiment, the blue sepharose chromatography may be used in a purification method having one or more additional steps, preferably selected from affinity chromatography, ion exchange chromatography, hydroxyapatite chromatography hydrophobic interaction chromatography or ultrafiltration.

In a highly preferred embodiment, the method of the invention is used as a second step of a purification scheme of an Fc-containing protein wherein the fluid loaded in step (a) on the blue sepharose resin is the eluate of Protein A or Protein G or Protein L affinity chromatography to which a fluid comprising said Fc-containing protein was subjected first.

In accordance with the present invention, a fluid comprising an Fc-containing protein is first subjected to Protein A or Protein G or Protein L or Protein NG affinity chromatography. The fluid may preferably be cell culture material, e.g. solubilized cells, more preferably cell culture supernatant.

The Protein A, G, NG or L used for the affinity chromatography may e.g. be recombinant. It may also be modified in order to improve its properties (such as e.g. in the resin called MabSelect SuRe, commercially available from GE Healthcare). In a preferred embodiment, the capture step is carried out on a resin comprising cross-linked agarose modified with recombinant Protein A. A column commercially available under the name MabSelect Xtra (from GE Healthcare) is an example of an affinity resin that is particularly suitable for step (a) of the present method.

The Protein A or G or L affinity chromatography is preferably used as a capture step, and thus serves for purification of the Fc-containing protein, in particular elimination of host cell proteins and Fc-containing protein aggregates, and for concentration of the Fc-containing protein preparation.

The elution of the Fc-containing protein in step (c) is carried out in a buffer with a pH ranging from about 4.0 to about 9. In a preferred embodiment, the elution is carried out in a buffer selected from sodium acetate or sodium citrate to which a salt is added. Suitable buffer concentrations are e.g. selected from about 25 mM, or about 50 mM or about 100 mM or about 150 mM or about 200 mM or about 250 mM. The salt concentration of the elution buffer in step (c) may be e.g. be 2.0, 2.1, 2.2, 2.3, 2.4, 2.5, 2.6, 2.7, 2.8, 2.9, 3.0 M KCl.

In accordance with the present invention, the eluate of the blue sepharose chromatography of step (c) is then used for further purification.

In a preferred embodiment of the invention, step (a) comprises loading the blue sepharose resin at a dynamic capacity of about 20 mg of Fc-containing protein per millilitre of packed blue sepharose resin. The resin is preferably loaded at pH 5.

In addition, the blue sepharose chromatography of the invention reduces the levels of free Fc-moieties to below detection levels as determined by SDS-PAGE. Therefore, in a preferred embodiment of the invention, the eluate of the blue dye chromatography has levels of free Fc-moieties, that are undetectable by SDS-PAGE under non-reducing conditions and silver staining when loading 1 mcg of Fc-containing protein.

The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

In a preferred embodiment of the present purification process, one or more ultrafiltration steps are performed. Ultrafiltration is useful for removal of small organic molecules and salts in the eluate resulting from previous chromatographic steps, to equilibrate the Fc-containing protein in the bulk buffer, or to concentrate the Fc-containing protein to the desired concentration. Such ultrafiltration may e.g. be performed on ultrafiltration membranes, with pore sizes allowing the removal of components having molecular weights below 5, 10, 15, 20, 25, 30 or more kDa.

If the protein purified according to the process of the invention is intended for administration to humans, it is advantageous to include one or more steps of virus removal in the process.

In order to facilitate storage or transport, for instance, the material may be frozen and thawed before and/or after any purification step of the invention.

In accordance with the present invention, the recombinant Fc-containing protein may be produced in eukaryotic expression systems, such as yeast, insect, or mammalian cells, resulting in glycosylated Fc-containing proteins.

In accordance with the present invention, it is most preferred to express the Fc-containing protein in mammalian cells such as animal cell lines, or in human cell lines. Chinese hamster ovary cells (CHO) or the murine myeloma cell line NS0 are examples of cell lines that are particularly suitable for expression of the Fc-containing protein to be purified. The Fc-containing protein can also preferably be produced in human cell lines, such as e.g. the human fibrosarcoma HT1080 cell line, the human retinoblastoma cell line PERC6, or the human embryonic kidney cell line 293, or a permanent amniocyte cell line as described e.g. in EP 1 230 354 .

If the Fc-containing protein to be purified is expressed by mammalian cells secreting it, the starting material of the purification process of the invention is cell culture supernatant, also called harvest or crude harvest. If the cells are cultured in a medium containing animal serum, the cell culture supernatant also contains serum proteins as impurities.

Preferably, the Fc-containing protein expressing and secreting cells are cultured under serum-free conditions. The Fc-containing protein may also be produced in a chemically defined medium. In this case, the starting material of the purification process of the invention is serum-free cell culture supernatant that mainly contains host cell proteins as impurities. If growth factors are added to the cell culture medium, such as insulin, for example, these proteins will be eliminated during the purification process as well.

In order to create soluble, secreted Fc-containing protein, that are released into the cell culture supernatant, either the natural signal peptide of the therapeutic moiety of the Fc-containing protein is used, or preferably a heterologous signal peptide, i.e. a signal peptide derived from another secreted protein being efficient in the particular expression system used, such as e.g. the bovine or human Growth Hormone signal peptide, or the immunoglobulin signal peptide.

As mentioned above, a preferred Fc-containing protein to be purified in accordance with the present invention is a fusion protein having a therapeutic moiety derived from human TACI (SwissProt entry O14836), and in particular a fragment derived from its extracellular domain (amino acids 1 to 165). In the following, therapeutic moieties derived from the extracellular domain of TACI will be called “soluble TACI” or “sTACI”. A preferred Fc-moiety comprises the Fc-fusion protein according to SEQ ID NO: 2, in the following called “TACI-Fc”. The term TACI-Fc, as used herein, also encompasses muteins of TACI-Fc.

The term “muteins”, as used herein, refers to analogs of sTACI, TACI-Fc or IFNβ-Fc in which one or more of the amino acid residues of sTACI, TACI-Fc or IFNβ-Fc are replaced by different amino acid residues, or are deleted, or one or more amino acid residues are added to the original sequence of sTACI, TACI-Fc or IFNβ-Fc without changing considerably the activity of the resulting products as compared with the original sTACI, r TACI-Fc or IFNβ-Fc. These muteins are prepared by known synthesis and/or by site-directed mutagenesis techniques, or any other known technique suitable therefore.

Muteins in accordance with the present invention include proteins encoded by a nucleic acid, such as DNA or RNA, which hybridizes to DNA or RNA, which encodes a TACI-Fc according to SEQ ID NO: 2 or IFNβ-Fc acoording to SEQ ID NO: 4 under stringent conditions. An example for a DNA sequence encoding a TACI-Fc is SEQ ID NO: 3 and an example encoding IFNβ-Fc is SEQ ID NO: 5.

The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992). Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990).

Any such mutein preferably has a sequence of amino acids sufficiently duplicative of that of e.g. sTACI or TACI-Fc, such as to have substantially similar ligand binding activity as a protein of SEQ ID NO: 2. For instance, one activity of TACI is its capability of binding to Blys or APRIL (Hymowitz et al., 2005). As long as the mutein has substantial APRIL or Blys binding activity, it can be considered to have substantially similar activity to TACI. Thus, it can be easily determined by the person skilled in the art whether any given mutein has substantially the same activity as a protein of SEQ ID NO: 2 by means of routine experimentation.

In a preferred embodiment, any such mutein has at least 50%, at least 60%, at least 70%, at least 75%, at least 80%, at least 85%, at least 90%, or at least 95% identity or homology thereto.

Preferred changes for muteins in accordance with the present invention are what are known as “conservative” substitutions. Conservative amino acid substitutions of sTACI or TACI-Fc, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, under twenty, or preferably under ten, and do not remove or displace amino acids which are critical to a functional conformation, e.g., cysteine residues. Proteins and muteins produced by such deletions and/or insertions come within the purview of the present invention.

Preferably, the conservative amino acid groups are those defined in Table 3. More preferably, the synonymous amino acid groups are those defined in Table 4; and most preferably the synonymous amino acid groups are those defined in Table 5.

TABLE 3
Preferred Groups of Synonymous Amino Acids

| Amino Acid | Synonymous Group |
|---|---|
| Ser | Thr, Gly, Asn |
| Arg | Gln, Lys, Glu, His |
| Leu | Ile, Phe, Tyr, Met, Val |
| Pro | Gly, Ala, Thr |
| Thr | Pro, Ser, Ala, Gly, His, Gln |
| Ala | Gly, Thr, Pro |
| Val | Met, Tyr, Phe, Ile, Leu |
| Gly | Ala, Thr, Pro, Ser |
| Ile | Met, Tyr, Phe, Val, Leu |
| Phe | Trp, Met, Tyr, Ile, Val, Leu |
| Tyr | Trp, Met, Phe, Ile, Val, Leu |
| Cys | Ser, Thr |
| His | Glu, Lys, Gln, Thr, Arg |
| Gln | Glu, Lys, Asn, His, Thr, Arg |
| Asn | Gln, Asp, Ser |
| Lys | Glu, Gln, His, Arg |
| Asp | Glu, Asn |
| Glu | Asp, Lys, Asn, Gln, His, Arg |
| Met | Phe, Ile, Val, Leu |

TABLE 4
More Preferred Groups of Synonymous Amino Acids

| Amino Acid | Synonymous Group |
|---|---|
| Arg | His, Lys |
| Leu | Ile, Phe, Met |
| Pro | Ala |
| Ala | Pro |
| Val | Met, Ile |
| Ile | Met, Phe, Val, Leu |
| Phe | Met, Tyr, Ile, Leu |
| Tyr | Phe |
| Cys | Ser |
| His | Gln, Arg |
| Gln | Glu, His |
| Asn | Asp |
| Lys | Arg |
| Asp | Asn |
| Glu | Gln |
| Met | Phe, Ile, Val, Leu |

TABLE 5
Most Preferred Groups of Synonymous Amino Acids

| Amino Acid | Synonymous Group |
|---|---|
| Leu | Ile, Met |
| Ile | Met, Leu |
| Cys | Ser |
| Met | Ile, Leu |
| Trp | Met |

“Functional derivatives” as used herein cover derivatives of the Fc-containing protein to be purified in accordance with the present invention, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of the unmodified Fc-containing protein as defined above, and do not confer toxic properties on compositions containing it.

Functional derivatives of an Fc-containing protein can e.g. be conjugated to polymers in order to improve the properties of the protein, such as the stability, half-life, bioavailability, tolerance by the human body, or immunogenicity. To achieve this goal, the Fc-containing protein may be linked e.g. to polyethylene glycol (PEG). PEGylation may be carried out by known methods, described in WO 92/13095, for example.

Functional derivatives may also, for example, include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

In a third aspect, the invention relates to a protein purified by the process of purification according to the invention. In the following, such protein is also called “purified Fc-containing protein”.

Such purified Fc-containing protein is preferably highly purified Fc-containing protein. Highly purified Fc-fusion protein is determined e.g. by the presence of a single band in a silver-stained, non-reduced SDS-PAGE-gel after loading of protein in the amount of 2 mcg per lane. Purified Fc-fusion protein may also be defined as eluting as a single peak in HPLC.

Purified Fc-containing protein may be intended for therapeutic use, in particular for administration to human patients. If purified Fc-containing protein is administered to patients, it is preferably administered systemically, and preferably subcutaneously or intramuscularly, or topically, i.e. locally. Rectal or intrathecal administration may also be suitable, depending on the specific medical use of purified Fc-containing protein.

For this purpose, in a preferred embodiment of the present invention, the purified Fc-containing protein may be formulated into pharmaceutical composition, i.e. together with a pharmaceutically acceptable carrier, excipients or the like.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective amounts of the active protein(s) will be a function of many variables, including the type of Fc-containing protein, the affinity of the Fc-containing protein for its ligand, the route of administration, the clinical condition of the patient.

A “therapeutically effective amount” is such that when administered, the Fc-containing protein results in inhibition of its ligand of the therapeutic moiety of the Fc-fusion protein, as explained above and referring particularly to Table 2 above.

The dosage administered, as single or multiple doses, to an individual will vary depending upon a variety of factors, including pharmacokinetic properties of the Fc-fusion protein, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of the natural ligand of the therapeutic moiety in an individual.

Purified Fc-containing protein may be used in an amount of about 0.001 to 100 mg/kg or about 0.01 to 10 mg/kg or body weight, or about 0.1 to 5 mg/kg of body weight or about 1 to 3 mg/kg of body weight or about 2 mg/kg of body weight.

In further preferred embodiments, the purified Fc-containing protein is administered daily or every other day or three times per week or once per week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

The present invention further relates to the use of blue sepharose affinity chromatography for the reduction of the concentration of free Fc-moieties in a composition comprising an Fc-containing protein.

In a preferred embodiment, the concentration of free Fc is reduced to less than about 5% or less than about 2% or less than about 1% or less than about 0.5% or less than about 0.2% or less than about 0.1% of the total protein concentration of said composition.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### Examples

### List of Abbreviations Frequently used throughout the Examples

BV: bed volume

CHO: Chinese Hamster Ovary

Cond.: Conductivity

HPLC: High Performance Liquid Chromatography

K: potassium

KCl: Potassium chloride

kD: kilo Dalton

MALDI-MS: Matrix Assisted Laser Desorption Ionisation-Mass Spectrometry

Na: sodium

NaCl: Sodium chloride

NaOH: Sodium hydroxide

NH4OH: Ammonium hydroxide

OD: Optical density

rh: Recombinant human

SDS-PAGE: Sodium Dodecyl Sulphate Polyacrylamide Gel Electrophoresis

UV: Ultra-Violet

### Example 1

### Purification of TACI-Fc via Blue Sepharose Chromatography (Gradient Elution)

Blue Sepharose chromatography was developed for the removal of free Fc fragments resulting from the cleavage of Fc-containing proteins.

Clarified harvest of a TACI-Fc (a homodimer Fc fusion protein, the amino sequence of each subunit corresponding to the amino acid sequence of SEQ ID: NO 2) expressing CHO cell clone cultured under serum-free conditions was subjected to Protein A affinity chromatography as capture step. The eluate from the capture step on Protein A was used as a starting material for the blue sepharose chromatography.

All the operations were performed at room temperature and the flow rate was kept constant at 175 cm/h. The UV signal at 280 nm was recorded at all time.

Column

Blue Sepharose 6FF resin (GE Healthcare) was packed into a 121 ml volume column of 3.2 cm internal diameter having a bed height of 15 cm.

Equilibration

The column was equilibrated with 5 BV of 25 mM sodium acetate pH 5.0.

Loading

The column was loaded with Protein A capture column eluate at pH 5 at a capacity of about 20 mg TACI-Fc per ml of packed resin.

Wash 1

The column was washed with at least 3 BV of 25 mM sodium acetate pH 5.0 until a stable baseline was reached.

Elution

Gradient elution was achieved with 20 BV of 0-3 M potassium chloride in 25 mM sodium acetate pH 5.0

Regeneration & Sanitisation

The column was sanitised with 5 BV of 0.5 M NaOH in reverse flow mode then washed with 5 BV of equilibration buffer before storage in 3 BV of 20% ethanol.

Different fractions were collected and subjected to SDS-PAGE analysis, Western immunoblotting, MALDI-MS spectrometry and N-terminal sequencing.

Results

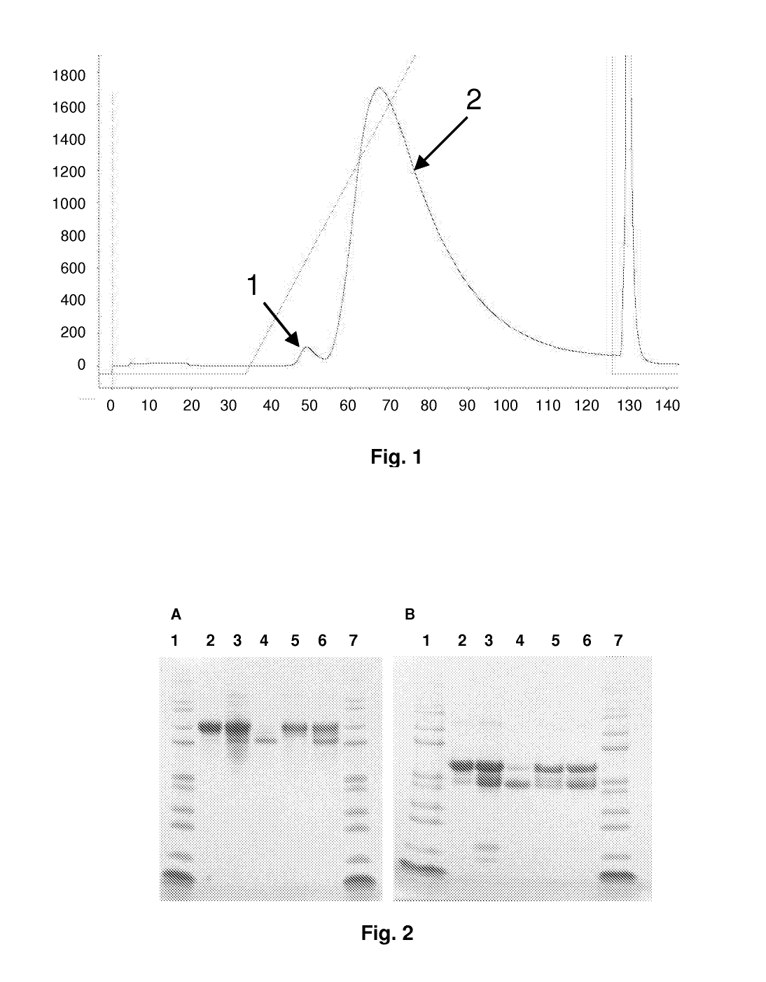

As shown in the chromatographic profile of FIG. 1, the Blue Sepharose resolved a separate peak during gradient elution. A separate peak, peak 1, was identified as free Fc with a molecular weight of about 55 kDa by SDS-PAGE, Western blot, MALDI-MS and N-terminal sequencing. This fragment results from the cleavage of the molecule between the amino acids arginine at position 80 and serine at position 81 of the TACI domain just before the junction with the hinge region. The second elution peak, peak 2, represents purified TACI-Fc.

SDS-Page Analysis

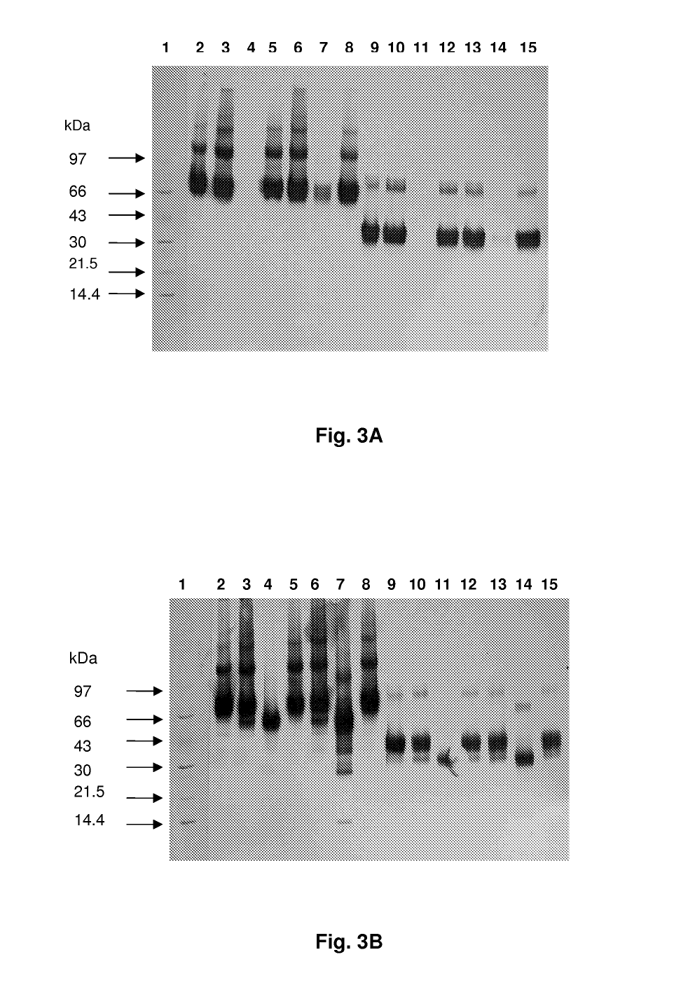

As shown in the silver stained SDS-PAGE gel under non-reducing conditions of FIG. 2A, a main band of intact TACI-Fc and a minor band of lower molecular weight protein corresponding to the Free Fc were visible in lane 3 corresponding to the load. In lane 4, corresponding to peak 1, only the lower molecular weight band (free Fc) was visible whereas in lane 5 (peak 2) a band corresponding to pure TACI-Fc without Free Fc contaminant was visible. Under reducing conditions (FIG. 2B) traces of Fc fragment corresponding to the hybrid TACI-Fc/Fc form were detected in peak 2 (lane 5).

Western Immunoblotting Analysis

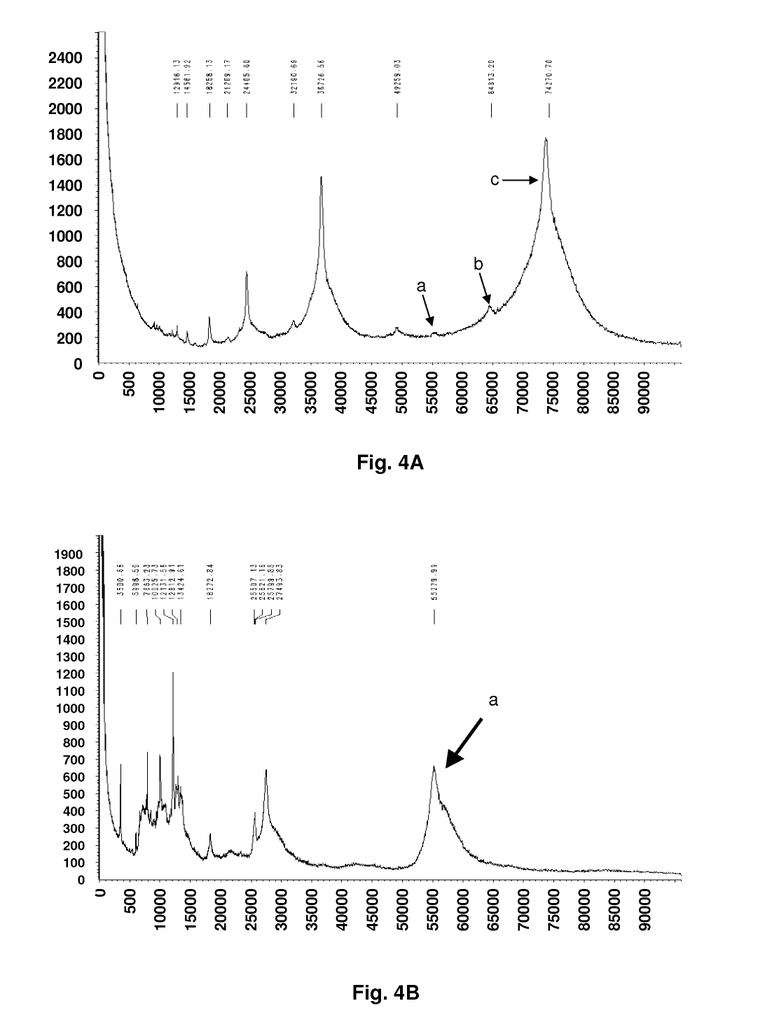

Following anti-TACI immunodetection under non-reducing conditions (FIG. 3A, lanes 2 to 5) and reducing conditions (FIG. 3A, lanes 9 to 12), no TACI domain was detected in peak 1 (lanes 4 and 11).

Following anti-Fc immunodetection under non-reducing conditions (FIG. 3B, lanes 2 to 5) and reducing conditions (FIG. 3B, lanes 9 to 12), Fc domain was detected as follows:
in peak 1 (lanes 4 and 11) as a band of low molecular weight (55 kDa) corresponding to free Fc.
in the load (lanes 3 and 10) as 2 bands of high and low molecular weight corresponding to intact TACI-Fc and free Fc respectively.
in peak 2 (Lanes 5 and 12) as a band of high molecular weight (73 kDa) corresponding to TACI-Fc.

Lanes 6 to 8 and 13 to 15 represent fractions stemming from a wash under different conditions not further specified.

MALDI-MS Analysis

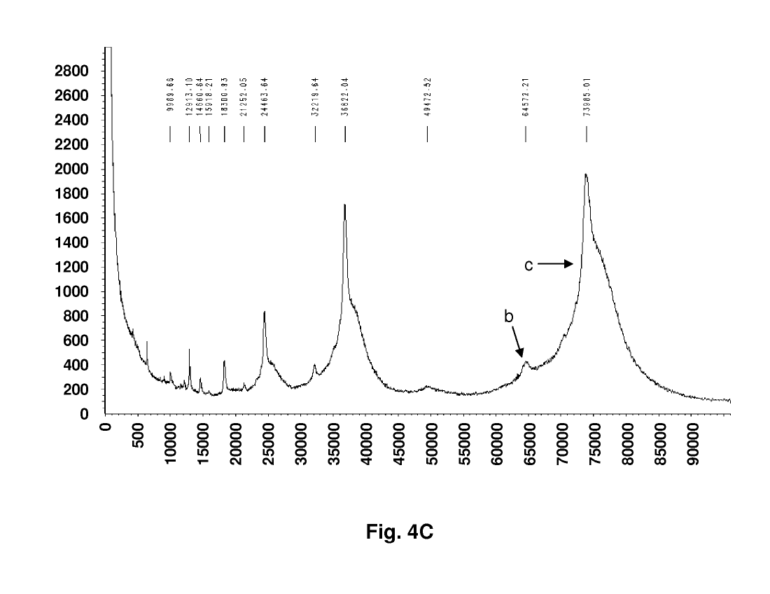

As shown in the MALDI-MS spectra in FIG. 4, Free Fc was identified as a peak (peak a) at about 55 kDa in the load (FIG. 4A) and in peak 1 (FIG. 4B) fractions but not in peak 2 fraction (FIG. 4C). Intact TACI-Fc was identified as a peak at about 74 kDa (peak b) in the load (FIG. 4A) and peak 2 (FIG. 4C) fractions. Hybrid TACI-Fc/Fc was identified as a peak at about 64.5 kDa in the load (FIG. 4A) and peak 2 (FIG. 4C) fractions.

N-Terminal Sequencing

In Peak 1, only a sequence starting at Ser81 was detected corresponding to Free Fc.

Conclusion

The TACI-Fc eluate from blue sepharose was found to be pure of Free Fc. Gradient elution of the blue sepharose column with 0-3 M potassium chloride in 25 mM sodium acetate pH5.0 resulted indeed in the resolution of two peaks representing the free Fc fraction (peak 1) and the purified TACI-Fc fraction (peak 2). TACI-Fc recovery over the Blue Sepharose step was >90%.

Gradient elution in 0-3 M KCl was also performed in 25 mM Na acetate at pH 4.5 and 5.5 with a complete resolution of the peak of Free Fc.

### Example 2

### Blue Sepharose Chromatography (Step Elution)—TACI-Fc

The same protocol as the one described in Example 1 was followed except for the gradient elution step which was replaced by a step elution (“Wash 2” and “Elution” steps) as follows:

Column

Blue Sepharose 6FF resin (GE Healthcare) was packed into a 121 ml volume column of 3.2 cm internal diameter having a bed height of 15 cm.

Equilibration

The column was equilibrated with 5 BV of 25 mM sodium acetate pH 5.0.

Loading

The column was loaded with Protein A capture column eluate at pH 5 at a capacity of about 20 mg TACI-Fc per ml of packed resin.

Wash 1

The column was washed with at least 3 BV of 25 mM sodium acetate pH 5.0 until a stable baseline was reached.

Wash 2

The column was washed with 5 BV of 25 mM sodium acetate+240 mM potassium chloride pH 5.0

Elution

Elution of TACI-Fc was achieved with 10 BV of 25 mM sodium acetate+2 M potassium chloride pH 5.0.

Regeneration & Sanitisation

The column was sanitised with 5 BV of 0.5 M NaOH in reverse flow mode then washed with 5 BV of equilibration buffer before storage in 3 BV of 20% ethanol.

Results

The results were the same as those obtained in Example 1. The resulting free Fc fraction was removed by a step elution in 25 mM sodium acetate, 240 mM potassium chloride at pH 5.0 while the intact TACI-Fc molecule was eluted with 25 mM sodium acetate, 2 M potassium chloride at pH 5.0.

### Example 3

### Blue Sepharose Chromatography—IFN-beta-Fc

Clarified harvest of an IFN-β-Fc expressing CHO cell clone cultured under serum-free conditions was subjected to Protein A affinity chromatography as capture step. IFN-β-Fc is an Fc-fusion fusion protein created by the fusion of an Interferon-beta protein and of an Fc domain. The Fc domain, also called Fc fragment or region, of an immunoglobulin consists of two identical arms which comprise the hinge region (H) and the second (CH2) and third (CH3) domain of an antibody heavy chain. The IFN-β-Fc contains two subunits, the first comprising a mutated IgG Fc arm linked to a single IFN-β protein (SEQ ID: NO 4) and the second subunit comprising a mutated IgG Fc arm (amino acids 167 to 393 of SEQ ID: NO 4). The eluate from the capture step on Protein A was first filtrated and further used as a starting material for the blue sepharose chromatography and the following steps followed:

| Step | Equilibration | Load | Wash 1 | Wash 2 | Wash 3 | Wash 4 | Wash 5 | Wash 6 | Elution | Regeneration |
|---|---|---|---|---|---|---|---|---|---|---|
| Buffer | 50 mM | NA | 50 mM | 50 mM | 50 mM | 50 mM | 50 mM | 50 mM | 1 M | NaOH |
| | Sodium | | Sodium | sodium | Sodium | Sodium | Sodium | Sodium | NH4OH | 0.5 M |
| | Acetate | | Acetate | acetate | Acetate | Acetate | Acetate | Acetate | | |
| pH | 5 | 5 | 5 | 5 | 5 | 5 | 5 | 5 | 12 | >12 |
| conductivity | 3.1 | 2.35 | 3.1 | 3.1 to 38 | 82 | 139 | 212 | 300 | NA | NA |
| mS/cm | | | | | | | | | | |
| [KCl] M | 0 | 0 | 0 | 0 to 0.5 | 1 | 1.5 | 2 | 3 | 0 | 0 |
| BV (at least) | 5 | NA | 4 | 22 | 5 | 6 | 8 | 13 | 3 | 4 |

After loading the Protein A eluate on the blue sepharose resin (At a dynamic capacity of 2.5 g/L IFN-Fc per of packed resin), washes were performed with 50 mM sodium acetate buffer at pH5 containing increasing levels of potassium chloride. Elution was carried out with 1M ammonium hydroxide at pH12.

All the operations were performed at room temperature and the flow rate was kept constant at 150 cm/h. The OD signal at 280 nm was recorded at all time.

Results

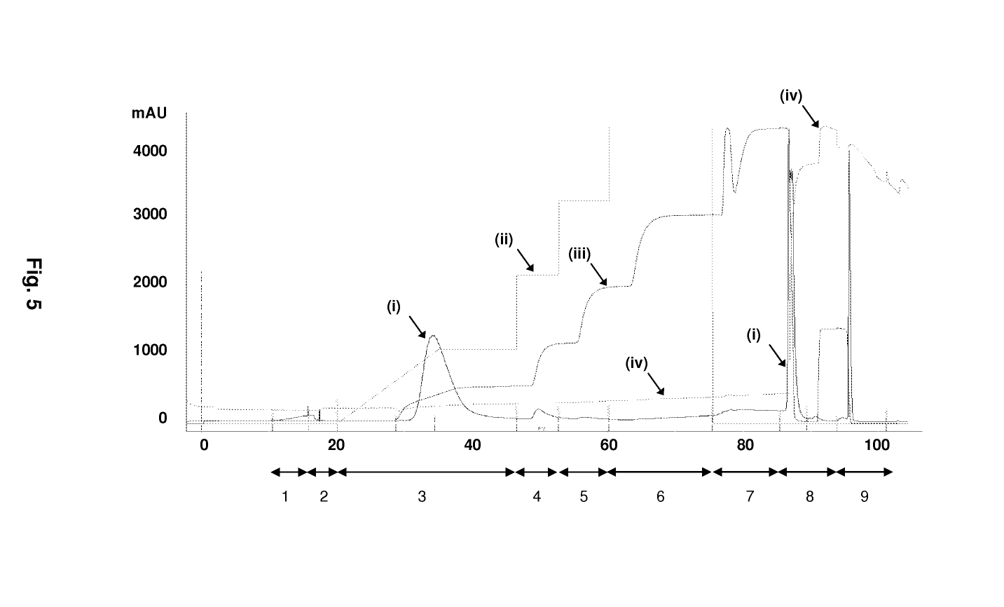

As shown in the chromatographic profile of FIG. 5, a peak eluting at wash step 2 i.e. when the column was washed with at least 22 BV of 50 mM sodium acetate and a gradient of potassium chloride from 0 to 500 mM at pH5 was identified as free Fc. The interferon domain having a very high affinity for the Cibacron Blue ligand, the purified IFN-Fc was eluted from the blue sepharose column with 1 M ammonium hydroxide at pH 12.

Overall Conclusion

Removal of Free Fc can be achieved either by a gradient (0-3 M KCl at pH 5.0) or by a step elution of the blue sepharose column at pH 4.5, pH 5.0 or pH 5.5. The step elution was optimised for TACI-Fc into a two step method comprising washing the column under conditions suitable for Free Fc removal i.e. washing the column post load with 240-300 mM KCl in 25 mM Na acetate at pH 5.0, followed by elution of TACI-Fc using another set of conditions gave optimal Free Fc removal without affecting intact TACI-Fc recovery. For IFN-β-Fc the free Fc fragment was removed in a wash step with 50 mM sodium acetate and a gradient of potassium chloride from 0 to 500 mM at pH 5 and IFNβ-Fc was then eluted with 1 M ammonium hydroxide at pH 12.

Therefore for both Fc fusion proteins tested, TACI-Fc and IFN-β-Fc, the free Fc fragment was eluted/washed under the same conditions from the blue sepharose column i.e. between pH 4.5 to 5.5.

### REFERENCES

Altschul, S. F., Gish, W., Miller, W., Myers, E. W., and Lipman, D. J. (1990). Basic local alignment search tool. J. Mol. Biol. 215, 403-410.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
Armour K L, Clark M R, Hadley A G, Williamson L M. (1999). Recombinant human IgG molecules lacking Fcgamma receptor I binding and monocyte triggering activities. Eur J Immunol. 29(8):2613-24
Ausubel et al., Current Protocols in Molecular Biology, supra, Interscience, N.Y., §§6.3 and 6.4 (1987, 1992).
Bodmer J L, Schneider P, Tschopp J. (2002). The molecular architecture of the TNF superfamily. Trends Biochem Sci. 2002 January;27(1):19-26.
Bossen C, Ingold K, Tardivel A, Bodmer J L, Gaide O, Hertig S, Ambrose C, Tschopp J, Schneider P. (2006). Interactions of tumor necrosis factor (TNF) and TNF receptor family members in the mouse and human. J Biol Chem. 2006 May 19;281(20):13964-71.
Carter P J. (2006). Potent antibody therapeutics by design. Nat Rev Immunol. 2006 May;6(5):343-57.
Devereux, J., Haeberli, P., and Smithies, O. (1984). A comprehensive set of sequence analysis programs for the VAX. Nucleic Acids Res. 12, 387-395.
EP 1230 354
Grantham, R. (1974). Amino acid difference formula to help explain protein evolution. Science 185, 862-864.
Gross et al., (2000). TACI and BCMA are receptors for a TNF homologue implicated in B-cell autoimmune disease. Nature 404(6781):995-9.
Hahne et al. (1998). APRIL, a new ligand of the tumor necrosis factor family, stimulates tumor cell growth. J Exp Med. 188(6):1185-90.
Hinton P R, Johlfs M G, Xiong J M, Hanestad K, Ong K C, Bullock C, Keller S, Tang M T, Tso J Y, Vasquez M, Tsurushita N. (2004). Engineered human IgG antibodies with longer serum half-lives in primates. J Biol Chem. 279(8):6213-6.
Hymowitz et al., (2005). Structures of APRIL-receptor complexes: like BCMA, TACI employs only a single cysteine-rich domain for high affinity ligand binding. J Biol Chem. 280(8):7218-27. Epub 2004 Nov. 12.
Idusogie E E. et al. (2000). Mapping of the C1q binding site on rituxan, a chimeric antibody with a human IgG1 Fc. J Immunol. 164(8):4178-84.
Idusogie E E. et al. (2001). Engineered antibodies with increased activity to recruit complement. J Immunol. 166(4):2571-5.
Kabat, E. A., Wu, T. T., Perry, H. M., Gottesman, K. S., and Foeller, C. (1991), Sequences of Proteins of Immunological Interest, 5th Ed., National Institutes of Health, Bethesda, Md.
Knight E Jr and Fahey. (1981). Human fibroblast interferon. An improved purification. J Biol Chem. 256(8):3609-11.
Locksley et al. (2001). The TNF and TNF receptor superfamilies: integrating mammalian biology. Cell 104, 487-501.
Melchers F. (2003) Actions of BAFF in B cell maturation and its effects on the development of autoimmune disease. Ann Rheum Dis. 62 Suppl 2:ii25-7
Moore et al., (1999). BLyS: member of the tumor necrosis factor family and B lymphocyte stimulator. Science. 285(5425):260-3.
Naismith J H, Sprang S R. (1998). Modularity in the TNF-receptor family. Trends Biochem Sci. 23(2):74-9. Review.
Novak et al. (2004). Expression of BCMA, TACI, and BAFF-R in multiple myeloma: a mechanism for growth and survival. Blood. 103(2):689-94. Epub 2003 Sep. 25.
Pearson, W. R. (1990). Rapid and sensitive sequence comparison with FASTP and FASTA. Methods Enzymol. 183, 63-98.
Shields R L. et al. (2001). High resolution mapping of the binding site on human IgG1 for Fc gamma RI, Fc gamma RII, Fc gamma RIII, and FcRn and design of IgG1 variants with improved binding to the Fc gamma R. J Biol Chem. 276(9):6591-604.
Smith, T. F. and Waterman, M. S. (1981). Identification of common molecular subsequences. J. Mol. Biol. 147, 195-197.
Steurer W, Nickerson P W, Steele A W, Steiger J, Zheng X X, Strom T B. (1995). Ex vivo coating of islet cell allografts with murine CTLA4/Fc promotes graft tolerance. J Immunol. 155(3):1165-74
Vaccaro C, Zhou J, Ober R J, Ward E S. (2005). Engineering the Fc region of immunoglobulin G to modulate in vivo antibody levels. Nat Biotechnol. 23(10):1283-8.
Vlatakis G, Skarpelis G, Stratidaki I, Bouriotis V, Clonis Y D. (1987). Dye-ligand chromatography for the resolution and purification of restriction endonucleases. Appl Biochem Biotechnol. 15(3):201-12.
von Bulow G U, Bram R J. NF-AT activation induced by a CAML-interacting member of the tumor necrosis factor receptor superfamily. Science. 1997 October 3;278(5335):138-41.
Xia et al. (2000). TACI is a TRAF-interacting receptor for TALL-1, a tumor necrosis factor family member involved in B cell regulation. J Exp Med. 2000 July 3;192(1):137-43.
WO 00/40716,
WO 02/094852
WO 2004/045512
WO 2005/001025
WO 91/03553
WO 92/13095
WO 94/06476
WO 97/13852
WO 98/23761
U.S. Pat. No. 5,969,102

1-28. (canceled)

29. A method for purifying an Fc-containing protein from free Fc-moieties present in a fluid comprising said Fc-containing protein, the method comprising the steps of:
(a) loading said fluid on a blue dye affinity chromatography resin;
(b) washing the resin with a buffer having a pH of about 4.0 to about 6.0 thereby eliminating the free Fc-moieties from the resin; and
(c) eluting the Fc-containing protein from the resin.

30. The method according to claim 29, wherein in step (b) the buffer comprises a salt selected from potassium chloride or sodium chloride.

31. The method according to claim 29, wherein in step (b) the Fc moieties are washed from the blue dye affinity chromatography resin with an increasing salt gradient from about 0 to about 0.5 M KCl.

32. The method according to claim 29, wherein in step (b) the Fe moieties are washed from the blue dye affinity chromatography column with an isocratic salt concentration ranging from 200 to 300 mM KCl.

33. The method according to claim 29, wherein in step (b) the buffer comprises sodium acetate at about 10 to about 100 mM.

34. The method according to claim 29, wherein the dye affinity chromatography of Step (a) is carried out with a resin having immobilized Cibacron Blue F3G-A.

35. The method according to claim 34, wherein said Cibacron Blue F3G-A is immobilized on agarose.

36. The method according to claim 35, wherein step (a) comprises loading the blue sepharose resin at a dynamic capacity of about 20 mg of Fe-containing protein per milliliter of packed blue sepharose resin.

37. The method according to claim 29, wherein the fluid in step (a) is loaded on the resin at pH 5.

38. The method according to claim 28, wherein the eluate of the blue dye affinity chromatography resin resulting from step (c) has levels of free Fc moieties that are undetectable by SDS-PAGE under non-reducing conditions and silver staining when loading 1 mcg of Fe-containing protein.

39. The method according to claim 28, wherein in step (a) the Fc-containing fluid is Protein A chromatography eluate.

40. The method according to claim 28, further comprising one or more step of affinity chromatography, ion exchange chromatography, hydroxyapatite chromatography, hydrophobic interaction chromatography or ultrafiltration.

41. The method according to claim 28, further comprising formulating the purified Fc-containing protein into a pharmaceutical composition.

42. The method according to claim 28, wherein the Fe-containing protein comprises an Immunoglobulin (Ig) constant region.

43. The method according to claim 42, wherein the constant region is a human constant region.

44. The method according to claim 42, wherein the immunoglobulin is an IgG₁.

45. The method according to claim 42, wherein the constant region comprises a CH2 and a CH3 domain.

46. The method according to claim 29, wherein the Fc-containing protein comprises an immunoglobulin variable region.

47. The method according to claim 46, wherein the Fc-containing protein is an antibody.

48. The method according to claim 29, wherein the Fe-containing protein is an Fe-fusion protein.

49. The method according to claim 48, wherein the Fe-fusion protein comprises a ligand binding portion of a member of the tumor necrosis factor receptor (TNFR) superfamily.

50. The method according to claim 49, wherein the ligand binding portion is selected from an extracellular domain of TNFR1, TNFR2, or a TNF binding fragment thereof.

51. The method according to claim 50, wherein the ligand binding portion of said Fc-fusion protein is selected from an extracellular domain of BAFF-R, BCMA, TACI, or a fragment thereof binding at least one of Blys or APRIL.

52. The method according claim 51, wherein the Fc-fusion protein comprises a polypeptide selected from:
(a) SEQ ID NO: 2;
(b) a polypeptide encoded by a polynucleotide hybridizing to the complement of SEQ ID NO: 3 under highly stringent conditions; or
(c) a mutein of (a) having at least 80% or 85% or 90% or 95% sequence identity to the polypeptide of (a);
wherein the polypeptide binds to at least one of Blys or APRIL.

53. The method according to claim 48, wherein the Fe-fusion protein comprises IFN-β.

54. The method according claim 53, wherein the Fc-fusion protein comprises a polypeptide selected from:
(a) SEQ ID NO: 4;
(b) amino acids 22 to 422 of SEQ ID NO: 4;
(c) a polypeptide encoded by a polynucleotide hybridizing to the complement of SEQ ID NO: 5 under highly stringent conditions; or
(d) a mutein of any of (a) or (b) having at least 80% or 85% or 90% or 95% sequence identity to the polypeptide of (a) or (b).
